(19) United States
(12) Patent Application Publication
(10) Pub. No.: US 2011/0288009 A1
(43) Pub. Date: Nov. 24, 2011

(54) LEPTIN AND LEPTIN ANALOG CONJUGATES AND USES THEREOF

(75) Inventors: Jean-Paul Castaigne, Mont-Royal (CA); Michel Demeule, Beaconsfield (CA); Dominique Boivin, Ste-Marthe-sur-le-lac (CA); Betty Lawrence, Bolton (CA); Christian Che, Longueuil (CA)

(73) Assignee: Angiochem Inc., Montreal, QC (CA)

(21) Appl. No.: 13/132,838

(22) PCT Filed: Dec. 7, 2009
(86) PCT No.: PCT/CA2009/001780

§ 371 Date: Aug. 9, 2011

Related U.S. Application Data
(60) Provisional application No. 61/200,947, filed on Dec. 5, 2008.
(60) Provisional application No. 61/178,837, filed on May 15, 2009.

(51) Int. Cl. A61K 38/22; C07K 19/00; C07H 21/00; A61P 3/04; A61P 9/12; A61P 3/06; A61P 3/00; A61P 3/08; A61P 9/00; C07K 14/575; A61P 3/10

(52) U.S. Cl. 514/4.8; 530/399; 530/324; 536/23.4; 514/5.8

(57) ABSTRACT

The present invention features a compound having the formula A-X-B, where A is peptide vector capable of enhancing transport of the compound across the blood-brain barrier or into particular cell types, X is a linker, and B is a leptin, a leptin analog, or OB receptor agonist. The compounds of the invention can be used to treat any disease in which increased amounts of leptin are desired, such as metabolic diseases including obesity and diabetes.

### BACKGROUND OF THE INVENTION

The invention relates to compounds including a leptin, leptin analog, or OB receptor agonist bound to a peptide vector and uses thereof.

Throughout the world, the prevalence of obesity is on the increase. There are over 300 million obese adults (Body Mass Index (BMI)>30), according to the World Health Organization, and 1.1 billion overweight people (BMI>25) worldwide. In the United States, more than half of adults are overweight (64.5 percent) and nearly one-third (30.5 percent) are obese. Obesity is associated with conditions such as type 2 diabetes, coronary artery disease, increased incidence of certain cancers, respiratory complications, and osteoarthritis. Being overweight or obese are well-recognized factors that reduce life expectancy and are estimated to cause 300,000 premature deaths each year in the U.S. Medical guidelines to treat obese patients advise changes in eating habits and increased physical activity. Some therapeutic agents exist to aid in the treatment of obesity, however, they cannot substitute for changes in lifestyle.

Because obesity and related disorders are believed to involve changes in the brain, and because treatments that affect neurotransmission are needed in treatment of obesity, therapeutics that act on the brain need to have the ability to enter the brain in order to be efficacious. The blood-brain barrier (BBB) is considered a major obstacle for the potential use of drugs for treating disorders of the central nervous system (CNS). The global market for CNS drugs was $68 billion in 2006, which was roughly half that of global market for cardiovascular drugs, even though in the United States, nearly twice as many people suffer from CNS disorders as from cardiovascular diseases. The reason for this imbalance is, in part, that more than 98% of all potential CNS drugs do not cross the BBB. In addition, more than 99% of worldwide CNS drug development is devoted solely to CNS drug discovery, and less than 1% is directed to CNS drug delivery. This may explain the lack of therapeutic options available for major neurological diseases.

The brain is shielded against potentially toxic substances by the presence of two barrier systems: the BBB and the blood-cerebrospinal fluid barrier (BCSFB). The BBB is considered to be the major route for the uptake of serum ligands since its surface area is approximately 5000-fold greater than that of BCSFB. The brain endothelium, which constitutes the BBB, represents the major obstacle for the use of potential drugs against many disorders of the CNS. As a general rule, only small lipophilic molecules may pass across the BBB, i.e., from circulating systemic blood to brain. Many drugs that have a larger size or higher hydrophobicity show high efficacy in CNS targets but are not efficacious in animals as these drugs cannot effectively cross the BBB. Thus, peptide and protein therapeutics are generally excluded from transport from blood to brain, owing to the negligible permeability of the brain capillary endothelial wall to these drugs. Brain capillary endothelial cells (BCECs) are closely sealed by tight junctions, possess few fenestrae and few endocytic vesicles as compared to capillaries of other organs. BCECs are surrounded by extracellular matrix, astrocytes, pericytes, and microglial cells. The close association of endothelial cells with the astrocyte foot processes and the basement membrane of capillaries are important for the development and maintenance of the BBB properties that permit tight control of blood-brain exchange.

Thus, there exists a need for improved delivery of anti-obesity therapeutics, such as leptin and leptin analogs, to the brain, as well as to other tissues.

### SUMMARY OF THE INVENTION

To improve transport of leptin across the BBB, we have developed compounds that include (a) a leptin, leptin analog, or OB receptor agonsist and (b) a peptide vector. These compounds are useful in treating any leptin-related disorder (e.g., obesity) where increased transport of the polypeptide therapeutic across the BBB or into particular cell types is desired. The peptide vector is capable of transporting the polypeptide therapeutic either across the blood-brain barrier (BBB) or into a particular cell type (e.g., liver, lung, kidney, spleen, and muscle). Surprisingly, we have shown that lower doses of the exemplary polypeptide therapeutic, leptin₁₁₆₋₁₃₀, when conjugated to a peptide vector as described herein, are more effective in reducing weight and food intake than the unconjugated agent. Because the conjugates are targeted across the BBB or to particular cell types, therapeutic efficacy can be achieved using lower doses or less frequent dosing as compared to an unconjugated leptin, leptin analog, or OB receptor agonist, thus reducing the severity of or incidence of side effects and/or increasing efficacy. The compound may also exhibit increased stability, improved pharmacokinetics, or reduced degradation in vivo, as compared to the unconjugated polypeptide therapeutic.

Accordingly, in a first aspect the invention features a compound having the formula:

A-X-B

where A is a peptide vector capable of being transported across the blood-brain barrier (BBB) or into a particular cell type (e.g., liver, lung, kidney, spleen, and muscle), X is a linker, and B is polypeptide therapeutic selected from the group consisting of leptin, a leptin analog, and an OB receptor agonist. The transport across the BBB or into the cell may be increased by at least 10%, 25%, 50%, 75%, 100%, 200%, 500%, 750%, 1000%, 1500%, 2000%, 5000%, or 10,000%. The compound may be substantially pure. The compound may be formulated with a pharmaceutically acceptable carrier (e.g., any described herein).

In another aspect, the invention features methods of making the compound A-X-B. In one embodiment, the method includes conjugating the peptide vector (A) to a linker (X), and conjugating the peptide vector-linker (A-X) to leptin, a leptin analog, or an OB receptor agonist (B), thereby forming the compound A-X-B. In another embodiment, the method includes conjugating B to the linker (X), and conjugating the X-B to a peptide vector (A), thereby forming the compound A-X-B. In another embodiment, the method includes conjugating the peptide vector (A) to a leptin, a letpin analog, or to an OB receptor (B), where either A or B optionally include a linker (X), to form the compound A-X-B.

In another aspect, the invention features a nucleic acid molecule that encodes the compound A-X-B, where the compound is a polypeptide. The nucleic acid molecule may be operably linked to a promoter and may be part of a nucleic acid vector. The vector may be in a cell, such as a prokaryotic cell (e.g., bacterial cell) or eukaryotic cell (e.g., yeast or mammalian cell, such as a human cell).

In another aspect, the invention features methods of making a compound of the formula A-X-B, where A-X-B is a polypeptide. In one embodiment, the method includes expressing a nucleic acid vector of the previous aspect in a cell to produce the polypeptide; and purifying the polypeptide.

In another aspect, the invention features a method of treating (e.g., prophylactically) a subject having a metabolic disorder. The method includes administering a compound of the first aspect in an amount sufficient to treat the disorder. The metabolic disorder may be diabetes (e.g., Type I or Type II), obesity, diabetes as a consequence of obesity, hyperglycemia, dyslipidemia, hypertriglyceridemia, syndrome X, insulin resistance, impaired glucose tolerance (IGT), diabetic dyslipidemia, hyperlipidemia, a cardiovascular disease, or hypertension.

In another aspect, the invention features a method of reducing food intake by, or reducing body weight of, a subject. The method includes administering a compound of the first aspect of the invention to a subject in an amount sufficient to reduce food intake or reduce body weight. The subject may be overweight, obese, or bulimic.

In any of the methods involving administration of a compound to a subject, the amount sufficient may be less than 90%, 75%, 50%, 40%, 30%, 20%, 15%, 10%, 5%, 4%, 3%, 2%, 1%, or 0.1% of the amount required for an equivalent dose of the polypeptide therapeutic (e.g., any described herein) when not conjugated to the peptide vector. The amount sufficient may reduce a side effect (e.g., vomiting, nausea, or diarrhea) as compared to administration of an effective amount of the polypeptide therapeutic when not conjugated to the peptide vector. The subject may be a mammal such as a human.

In any of the above aspects, the peptide vector may be a polypeptide substantially identical to any of the sequences set Table 1, or a fragment thereof. In certain embodiments, the peptide vector has a sequence of Angiopep-1 (SEQ ID NO:67), Angiopep-2 (SEQ ID NO:97), Angiopep-3 (SEQ ID NO:107), Angiopep-4-a (SEQ ID NO:108), Angiopep-4-b (SEQ ID NO:109), Angiopep-5 (SEQ ID NO:110), Angiopep-6 (SEQ ID NO:111), or Angiopep-7 (SEQ ID NO:112)). The peptide vector or conjugate may be efficiently transported into a particular cell type (e.g., any one, two, three, four, or five of liver, lung, kidney, spleen, and muscle) or may cross the mammalian BBB efficiently (e.g., Angiopep-1, -2, -3, -4a, -4b, -5, and -6). In another embodiment, the peptide vector or conjugate is able to enter a particular cell type (e.g., any one, two, three, four, or five of liver, lung, kidney, spleen, and muscle) but does not cross the BBB efficiently (e.g., a conjugate including Angiopep-7). The peptide vector may be of any length, for example, at least 6, 7, 8, 9, 10, 11, 12, 13, 14, 15, 16, 17, 18, 19, 20, 21, 25, 35, 50, 75, 100, 200, or 500 amino acids, or any range between these numbers. In certain embodiments, the peptide vector is 10 to 50 amino acids in length. The polypeptide may be produced by recombinant genetic technology or chemical synthesis.

TABLE 1
Exemplary Peptide Vectors

| SEQ | |
|---|---|
| ID | |
| NO: | |
| 1 | T F V Y G G C R A K R N N F K S A E D |
| 2 | T F Q Y G G C M G N G N N F V T E K E |
| 3 | P F F Y G G C G G N R N N F D T E E Y |
| 4 | S F Y Y G G C L G N K N N Y L R E E E |
| 5 | T F F Y G G C R A K R N N F K R A K Y |
| 6 | T F F Y G G C R G K R N N F K R A K Y |
| 7 | T F F Y G G C R A K K N N Y K R A K Y |
| 8 | T F F Y G G C R G K K N N F K R A K Y |
| 9 | T F Q Y G G C R A K R N N F K R A K Y |
| 10 | T F Q Y G G C R G K K N N F K R A K Y |
| 11 | T F F Y G G C L G K R N N F K R A K Y |
| 12 | T F F Y G G S L G K R N N F K R A K Y |
| 13 | P F F Y G G C G G K K N N F K R A K Y |
| 14 | T F F Y G G C R G K G N N Y K R A K Y |
| 15 | P F F Y G G C R G K R N N F L R A K Y |
| 16 | T F F Y G G C R G K R N N F K R E K Y |
| 17 | P F F Y G G C R A K K N N F K R A K E |
| 18 | T F F Y G G C R G K R N N F K R A K D |
| 19 | T F F Y G G C R A K R N N F D R A K Y |
| 20 | T F F Y G G C R G K K N N F K R A E Y |
| 21 | P F F Y G G C G A N R N N F K R A K Y |
| 22 | T F F Y G G C G G K K N N F K T A K Y |
| 23 | T F F Y G G C R G N R N N F L R A K Y |
| 24 | T F F Y G G C R G N R N N F K T A K Y |
| 25 | T F F Y G G S R G N R N N F K T A K Y |
| 26 | T F F Y G G C L G N G N N F K R A K Y |
| 27 | T F F Y G G C L G N R N N F L R A K Y |
| 28 | T F F Y G G C L G N R N N F K T A K Y |
| 29 | T F F Y G G C R G N G N N F K S A K Y |
| 30 | T F F Y G G C R G K K N N F D R E K Y |
| 31 | T F F Y G G C R G K R N N F L R E K E |
| 32 | T F F Y G G C R G K G N N F D R A K Y |
| 33 | T F F Y G G S R G K G N N F D R A K Y |
| 34 | T F F Y G G C R G N G N N F V T A K Y |
| 35 | P F F Y G G C G G K G N N Y V T A K Y |
| 36 | T F F Y G G C L G K G N N F L T A K Y |
| 37 | S F F Y G G C L G N K N N F L T A K Y |
| 38 | T F F Y G G C G G N K N N F V R E K Y |
| 39 | T F F Y G G C M G N K N N F V R E K Y |
| 40 | T F F Y G G S M G N K N N F V R E K Y |
| 41 | P F F Y G G C L G N R N N Y V R E K Y |
| 42 | T F F Y G G C L G N R N N F V R E K Y |
| 43 | T F F Y G G C L G N K N N Y V R E K Y |
| 44 | T F F Y G G C G G N G N N F L T A K Y |
| 45 | T F F Y G G C R G N R N N F L T A E Y |
| 46 | T F F Y G G C R G N G N N F K S A E Y |
| 47 | P F F Y G G C L G N K N N F K T A E Y |
| 48 | T F F Y G G C R G N R N N F K T E E Y |
| 49 | T F F Y G G C R G K R N N F K T E E D |
| 50 | P F F Y G G C G G N G N N F V R E K Y |
| 51 | S F F Y G G C M G N G N N F V R E K Y |
| 52 | P F F Y G G C G G N G N N F L R E K Y |
| 53 | T F F Y G G C L G N G N N F V R E K Y |
| 54 | S F F Y G G C L G N G N N Y L R E K Y |
| 55 | T F F Y G G S L G N G N N F V R E K Y |
| 56 | T F F Y G G C R G N G N N F V T A E Y |
| 57 | T F F Y G G C L G K G N N F V S A E Y |
| 58 | T F F Y G G C L G N R N N F D R A E Y |
| 59 | T F F Y G G C L G N R N N F L R E E Y |
| 60 | T F F Y G G C L G N K N N Y L R E E Y |
| 61 | P F F Y G G C G G N R N N Y L R E E Y |
| 62 | P F F Y G G S G G N R N N Y L R E E Y |
| 63 | M R P D F C L E P P Y T G P C V A R I |
| 64 | A R I I R Y F Y N A K A G L C Q T F V Y G |
| 65 | Y G G C R A K R N N Y K S A E D C M R T C G |
| 66 | P D F C L E P P Y T G P C V A R I I R Y F Y |
| 67 | T F F Y G G C R G K R N N F K T E E Y |
| 68 | K F F Y G G C R G K R N N F K T E E Y |
| 69 | T F Y Y G G C R G K R N N Y K T E E Y |
| 70 | T F F Y G G S R G K R N N F K T E E Y |
| 71 | C T F F Y G C C R G K R N N F K T E E Y |
| 72 | T F F Y G G C R G K R N N F K T E E Y C |
| 73 | C T F F Y G S C R G K R N N F K T E E Y |
| 74 | T F F Y G G S R G K R N N F K T E E Y C |
| 75 | P F F Y G G C R G K R N N F K T E E Y |
| 76 | T F F Y G G C R G K R N N F K T K E Y |
| 77 | T F F Y G G K R G K R N N F K T E E Y |
| 78 | T F F Y G G C R G K R N N F K T K R Y |
| 79 | T F F Y G G K R G K R N N F K T A E Y |
| 80 | T F F Y G G K R G K R N N F K T A G Y |
| 81 | T F F Y G G K R G K R N N F K R E K Y |
| 82 | T F F Y G G K R G K R N N F K R A K Y |
| 83 | T F F Y G G C L G N R N N F K T E E Y |
| 84 | T F F Y G C G R G K R N N F K T E E Y |
| 85 | T F F Y G G R C G K R N N F K T E E Y |
| 86 | T F F Y G G C L G N G N N F D T E E E |
| 87 | T F Q Y G G C R G K R N N F K T E E Y |
| 88 | Y N K E F G T F N T K G C E R G Y R F |
| 89 | R F K Y G G C L G N M N N F E T L E E |
| 90 | R F K Y G G C L G N K N N F L R L K Y |
| 91 | R F K Y G G C L G N K N N Y L R L K Y |
| 92 | K T K R K R K K Q R V K I A Y E E I F K N Y |
| 93 | K T K R K R K K Q R V K I A Y |
| 94 | R G G R L S Y S R R F S T S T G R |
| 95 | R R L S Y S R R R F |
| 96 | R Q I K I W F Q N R R M K W K K |
| 97 | T F F Y G G S R G K R N N F K T E E Y |
| 98 | M R P D F C L E P P Y T G P C V A R I |
| | I R Y F Y N A K A G L C Q T F V Y G G |
| | C R A K R N N F K S A E D C M R T C G G A |
| 99 | T F F Y G G C R G K R N N F K T K E Y |
| 100 | R F K Y G G C L G N K N N Y L R L K Y |
| 101 | T F F Y G G C R A K R N N F K R A K Y |
| 102 | N A K A G L C Q T F V Y G G C L A K R N N F |
| | E S A E D C M R T C G G A |
| 103 | Y G G C R A K R N N F K S A E D C M R T C G |
| | G A |
| 104 | G L C Q T F V Y G G C R A K R N N F K S A E |
| 105 | L C Q T F V Y G G C E A K R N N F K S A |
| 107 | T F F Y G G S R G K R N N F K T E E Y |
| 108 | R F F Y G G S R G K R N N F K T E E Y |
| 109 | R F F Y G G S R G K R N N F K T E E Y |
| 110 | R F F Y G G S R G K R N N F R T E E Y |
| 111 | T F F Y G G S R G K R N N F R T E E Y |
| 112 | T F F Y G G S R G R R N N F R T E E Y |
| 113 | C T F F Y G G S R G K R N N F K T E E Y |
| 114 | T F F Y G G S R G K R N N F K T E E Y C |
| 115 | C T F F Y G G S R G R R N N F R T E E Y |
| 116 | T F F Y G G S R G R R N N F R T E E Y C |

Polypeptides Nos. 5, 67, 76, and 91, include the sequences of SEQ ID NOS: 5, 67, 76, and 91, respectively, and are amidated at the C-terminus.
Polypeptides Nos. 107, 109, and 110 include the sequences of SEQ ID NOS: 97, 109, and 110, respectively, and are acetylated at the N-terminus.

In any of the above aspects, the peptide vector may include an amino acid sequence having the formula:

X1-X2-X3-X4-X5-X6-X7-X8-X9-X10-X11-X12-X13-
X14-X15-X16-X17-X18-X19

where each of X1-X19 (e.g., X1-X6, X8, X9, X11-X14, and X16-X19) is, independently, any amino acid (e.g., a naturally occurring amino acid such as Ala, Arg, Asn, Asp, Cys, Gln, Glu, Gly, His, Ile, Leu, Lys, Met, Phe, Pro, Ser, Thr, Trp, Tyr, and Val) or absent and at least one (e.g., 2 or 3) of X1, X10, and X15 is arginine. In some embodiments, X7 is Ser or Cys; or X10 and X15 each are independently Arg or Lys. In some embodiments, the residues from X1 through X19, inclusive, are substantially identical to any of the amino acid sequences of any one of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, at least one (e.g., 2, 3, 4, or 5) of the amino acids X1-X19 is Arg. In some embodiments, the polypeptide has one or more additional cysteine residues at the N-terminal of the polypeptide, the C-terminal of the polypeptide, or both.

In certain embodiments of any of the above aspects, the peptide vector or leptin, leptin analog, or OB receptor agonist is modified (e.g., as described herein). The peptide vector or polypeptide therapeutic may be amidated, acetylated, or both. Such modifications may be at the amino or carboxy terminus of the polypeptide. The peptide vector or polypeptide therapeutic may also include or be a peptidomimetic (e.g., those described herein) of any of the polypeptides described herein. The peptide vector or polypeptide therapeutic may be in a multimeric form, for example, dimeric form (e.g., formed by disulfide bonding through cysteine residues).

In certain embodiments, the peptide vector or leptin, leptin analog, or OB receptor agonist has an amino acid sequence described herein with at least one amino acid substitution (e.g., 2, 3, 4, 5, 6, 7, 8, 9, 10, 11, or 12 substitutions), insertion, or deletion. The polypeptide may contain, for example, 1 to 12, 1 to 10, 1 to 5, or 1 to 3 amino acid substitutions, for example, 1 to 10 (e.g., to 9, 8, 7, 6, 5, 4, 3, 2) amino acid substitutions. The amino acid substitution(s) may be conservative or non-conservative. For example, the peptide vector may have an arginine at one, two, or three of the positions corresponding to positions 1, 10, and 15 of the amino acid sequence of any of SEQ ID NO:1, Angiopep-1, Angiopep-2, Angiopep-3, Angiopep-4a, Angiopep-4b, Angiopep-5, Angiopep-6, and Angiopep-7. In certain embodiments, the leptin, leptin analog, or agonist may have a cysteine or lysine substitution or addition at any position (e.g., a lysine substitution at the N- or C-terminal position).

In any of the above aspects, the compound may specifically exclude a polypeptide including or consisting of any of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, the polypeptides and conjugates of the invention exclude the polypeptides of SEQ ID NOs:102, 103, 104, and 105.

In any of the above aspects, the linker (X) may be any linker known in the art or described herein. In particular embodiments, the linker is a covalent bond (e.g., a peptide bond), a chemical linking agent (e.g., those described herein), an amino acid or a peptide (e.g., 2, 3, 4, 5, 8, 10, or more amino acids). In certain embodiments, the linker has the formula:

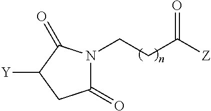

where n is an integer between 2 and 15 (e.g., 2, 3, 4, 5, 6, 7, 8, 9, 10, 11, 12, 13, 14, or 15); and either Y is a thiol on A and Z is a primary amine on B or Y is a thiol on B and Z is a primary amino on A.

In certain embodiments, the compound is a fusion protein including the peptide vector (e.g., Angiopep-2) and the polypeptide therapeutic (e.g., human leptin).

In any of the above embodiments, B may be leptin(116-130), leptin(22-56), leptin(57-92), leptin(93-105), LY396623, metreleptin, murine leptin analog, pegylated leptin, and methionyl human leptin. Resistins include human, mouse, and rat resistin. The leptin may be a mature sequence (e.g., amino acids 22-167 of the human sequence, e.g., shown in FIG. 16) or the full-length protein (e.g., shown in FIG. 16). The polypeptide used in the invention may be any of these peptides or may be substantially identical to any of these polypeptides.

By “peptide vector” is meant a compound or molecule such as a polypeptide or a polypeptide mimetic that can be transported into a particular cell type (e.g., liver, lungs, kidney, spleen, or muscle) or across the BBB. In certain embodiments, the vector may bind to receptors present on cancer cells or brain endothelial cells and thereby be transported into the cancer cell or across the BBB by transcytosis. The vector may be a molecule for which high levels of transendothelial transport may be obtained, without affecting the cell or BBB integrity. The vector may be a polypeptide or a peptidomimetic and may be naturally occurring or produced by chemical synthesis or recombinant genetic technology.

By “treating” a disease, disorder, or condition in a subject is meant reducing at least one symptom of the disease, disorder, or condition by administrating a therapeutic agent to the subject.

By “treating prophylactically” a disease, disorder, or condition in a subject is meant reducing the frequency of occurrence of or reducing the severity of a disease, disorder or condition by administering a therapeutic agent to the subject prior to the onset of disease symptoms.

In one example, a subject who is being treated for a metabolic disorder is one who a medical practitioner has diagnosed as having such a condition. Diagnosis may be performed by any suitable means, such as those described herein. A subject in whom the development of diabetes or obesity is being treated prophylactically may or may not have received such a diagnosis. One in the art will understand that subject of the invention may have been subjected to standard tests or may have been identified, without examination, as one at high risk due to the presence of one or more risk factors, such as family history, obesity, particular ethnicity (e.g., African Americans and Hispanic Americans), gestational diabetes or delivering a baby that weighs more than nine pounds, hypertension, having a pathological condition predisposing to obesity or diabetes, high blood levels of triglycerides, high blood levels of cholesterol, presence of molecular markers (e.g., presence of autoantibodies), and age (over 45 years of age). An individual is considered obese when their weight is 20% (25% in women) or more over the maximum weight desirable for their height. An adult who is more than 100 pounds overweight, is considered to be morbidly obese. Obesity is also defined as a body mass index (BMI) over 30 kg/m².

By “a metabolic disorder” is meant any pathological condition resulting from an alteration in a subject's metabolism. Such disorders include those resulting from an alteration in glucose homeostasis resulting, for example, in hyperglycemia. According to this invention, an alteration in glucose levels is typically an increase in glucose levels by at least 5%, 10%, 20%, 30%, 40%, 50%, 60%, 70%, 80%, 90%, or even 100% relative to such levels in a healthy individual. Metabolic disorders include obesity and diabetes (e.g., diabetes type I, diabetes type II, MODY, and gestational diabetes), satiety, and endocrine deficiencies of aging.

By “reducing glucose levels” is meant reducing the level of glucose by at least 10%, 20%, 30%, 40%, 50%, 60%, 70%, 80%, 90%, 95%, or 100% relative to an untreated control. Desirably, glucose levels are reduced to normoglycemic levels, i.e., between 150 to 60 mg/dL, between 140 to 70 mg/dL, between 130 to 70 mg/dL, between 125 to 80 mg/dL, and preferably between 120 to 80 mg/dL. Such reduction in glucose levels may be obtained by increasing any one of the biological activities associated with the clearance of glucose from the blood (e.g., increase insulin production, secretion, or action).

By “subject” is meant a human or non-human animal (e.g., a mammal).

By “equivalent dosage” is meant the amount of a compound of the invention required to achieve the same molar amount of the polypeptide therapeutic (e.g., leptin) in the compound of the invention, as compared to the unconjugated polypeptide therapeutic.

By a polypeptide which is “efficiently transported across the BBB” is meant a polypeptide that is able to cross the BBB at least as efficiently as Angiopep-6 (i.e., greater than 38.5% that of Angiopep-1 (250 nM) in the in situ brain perfusion assay described in U.S. patent application Ser. No. 11/807,597, filed May 29, 2007, hereby incorporated by reference). Accordingly, a polypeptide which is “not efficiently transported across the BBB” is transported to the brain at lower levels (e.g., transported less efficiently than Angiopep-6).

By a polypeptide or compound which is “efficiently transported to a particular cell type” is meant that the polypeptide or compound is able to accumulate (e.g., either due to increased transport into the cell, decreased efflux from the cell, or a combination thereof) in that cell type to at least a 10% (e.g., 25%, 50%, 100%, 200%, 500%, 1,000%, 5,000%, or 10,000%) greater extent than either a control substance, or, in the case of a conjugate, as compared to the unconjugated agent. Such activities are described in detail in International Application Publication No. WO 2007/009229, hereby incorporated by reference.

Other features and advantages of the invention will be apparent from the following Detailed Description, the drawings, and the claims.

### BRIEF DESCRIPTION OF THE DRAWINGS

FIGS. 1A and 1B are chromatograms showing the Leptin-AN2 (C11) conjugate before (FIG. 1A) and after (FIG. 1B) purification.

FIG. 2 is a chromatogram showing the results of purification of the Leptin-AN2 (C11) conjugate.

FIG. 3 is a graph showing uptake of the C3, C6, and C11 Leptin-AN2 conjugates into the brain, capillaries, and parenchyma using the in situ brain perfusion assay.

FIGS. 4A and 4B are graphs showing in situ brain perfusion of the leptin₁₁₆₋₁₃₀ and the Leptin-AN2 (C11) conjugate in lean mice and diet induced obese (DIO) mice (FIG. 4A) and plasma levels of leptin in lean mice and DIO mice (FIG. 4B).

FIGS. 5A and 5B are graphs showing food intake in mice receiving a control injection (saline), leptin₁₁₆₋₁₃₀ or the Leptin-AN2 (C11) conjugate after either four hours (FIG. 5A) or 15 hours (FIG. 5B).

FIG. 6 is a graph showing weight gain over a six-day period in mice receiving a control, leptin₁₁₆₋₁₃₀, or the Leptin-AN2 (C11) conjugate.

FIG. 7 is a graph showing weight gain over a ten-day period in ob/ob mice receiving a control, leptin₁₁₆₋₁₃₀, or the leptin-AN2 (C11) conjugate by daily IP injection over a period of six days.

FIG. 8 is a schematic diagram showing the GST tagged Angiopep construct.

FIG. 9 is a schematic diagram showing the PCR strategy used to generate the Angiopep-2-leptin₁₁₆₋₁₃₀ fusion protein.

FIG. 10 is a chromatogram showing purification of the GST-Angiopep2 on a GSH-sepharose column

FIGS. 11A-11C show a western blot (FIG. 11A), a UV spectrum from a liquid chromatography experiment (FIG. 11B), and a mass spectrum (FIG. 11C) of the recombinant Angiopep-2 peptide.

FIG. 12 is a graph showing uptake of the synthetic and recombinant forms of Angiopep-2 in the in situ brain perfusion assay.

FIG. 13 is a graph showing uptake of GST, GST-Angiopep-2, GST-leptin₁₁₆₋₁₃₀, and GST-Angiopep-2-leptin₁₁₆₋₁₃₀ into the parenchyma in the in situ brain perfusion assay.

FIG. 14 is a schematic diagram showing the His-tagged-mouse leptin and His-tagged-Angiopep-2-mouse leptin fusion protein.

FIG. 15 is an image of a gel showing purification of the His-tagged mouse leptin and the human leptin sequence.

FIG. 16 is the sequence of human leptin precursor. Amino acids 22-167 of this sequence form the mature leptin peptide.

FIGS. 17A and 17B are exemplary purification schemes for His-tagged leptin (mouse) and the His-tagged Angiopep-2-leptin conjugate.

FIG. 18 is photograph of a gel showing successful small-scale expression of the leptin and Angiopep-2-leptin conjugate.

FIG. 19 is a schematic diagram and picture of a gel showing that two products resulted from thrombing cleavage of the His-tagged conjugate.

FIG. 20 is a graph showing uptake of leptin and the Angiopep-2-leptin fusion protein into the parenchyma of DIO mice.

FIG. 21 is a graph showing the effect of recombinant leptin on the weight of ob/ob mice.

FIG. 22 is a graph showing the change in weight in DIO mice receiving a control, leptin, His-tagged mouse letpin, or the His-tagged Angiopep-2-leptin conjugate.

### DETAILED DESCRIPTION

We have developed polypeptide therapeutic conjugates having an enhanced ability to cross the blood-brain barrier (BBB) or to enter particular cell type(s) (e.g., liver, lung, kidney, spleen, and muscle) as exemplified by conjugates of peptide vectors to the exemplary polypeptide therapeutic, leptin. These exemplary polypeptide therapeutics can act as OB-R receptor agonists. The conjugates of the invention thus include a therapeutic polypeptide and a peptide vector that enhance transport across the BBB.

Surprisingly, we have shown that compounds of the invention, as compared to unconjugated forms of leptin, are more effective in reducing body weight. Greater efficacy can therefore lead to lower doses, fewer dosings, more effective treatments, or fewer side effects, as compared to the unconjugated polypeptide. Alternatively, increased efficacy at higher doses may be obtained.

### Leptin and Leptin Analogs

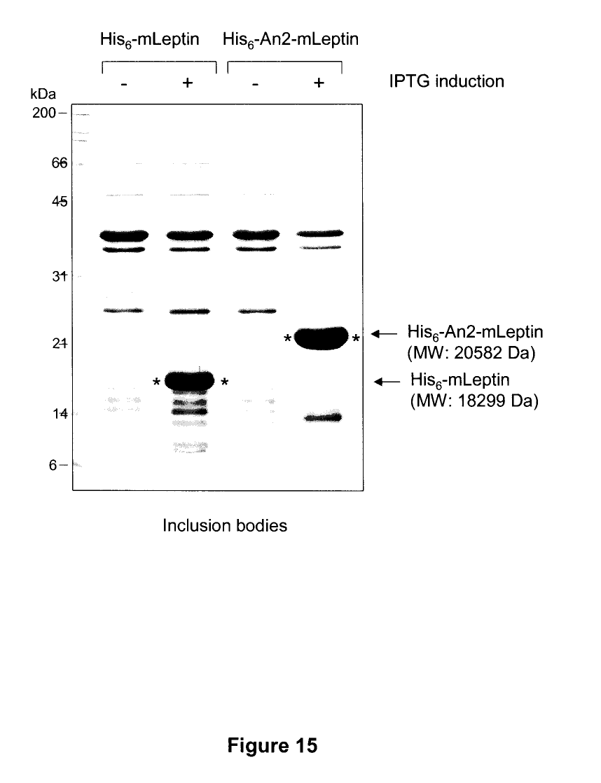

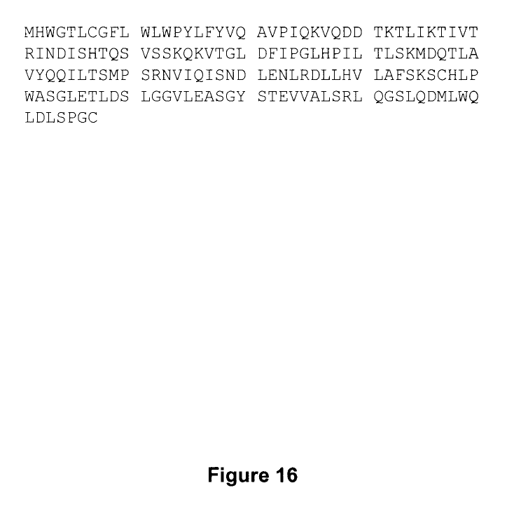

Leptin is an adipokine, and thus the proteins or peptides used in the invention can include an adipokine or an analog thereof. Adipokines include adiponectin, leptin, and resistin. Adiponectins include human, mouse, and rat adiponectin. Leptins include leptin(116-130), leptin(22-56), leptin(57-92), leptin(93-105), LY396623, metreleptin, murine leptin analog, pegylated leptin, and methionyl human leptin. Resistins include human, mouse, and rat resistin. The leptin may be a cleaved sequence (e.g., amino acids 22-167 of the human sequence, e.g., shown in FIG. 15) or the full length protein (e.g., shown in FIG. 15). The polypeptide used in the invention may be any of these peptides or proteins or may be substantially identical to any of these peptides or proteins.

The leptin analog may be an OB receptor agonist. In certain embodiments, the OB receptor agonist is an agonist for the OB-Rb form, which is the predominant receptor found in the hypothalamus or the OB-R, which is found at the blood-brain barrier and is involved in leptin transport.

### Modified Forms of Polypeptide Therapeutics

Any of the leptins, leptin analogs, or OB receptor agonists described herein may be modified (e.g., as described herein or as known in the art). As described in U.S. Pat. No. 6,924,264, the polypeptide can be bound to a polymer to increase its molecular weight. Exemplary polymers include polyethylene glycol polymers, polyamino acids, albumin, gelatin, succinyl-gelatin, (hydroxypropyl)-methacrylamide, fatty acids, polysaccharides, lipid amino acids, and dextran.

In one case, the polypeptide is modified by addition of albumin (e.g., human albumin), or an analog or fragment thereof, or the Fc portion of an immunoglobulin. Such an approach is described, for example, in U.S. Pat. No. 7,271,149.

In one example, the polypeptide is modified by addition of a lipophilic substituent, as described in PCT Publication WO 98/08871. The lipophilic substituent may include a partially or completely hydrogenated cyclopentanophenathrene skeleton, a straight-chain or branched alkyl group; the acyl group of a straight-chain or branched fatty acid (e.g., a group including CH₃(CH₂)ₙCO— or HOOC(CH₂)ₙCO—, where n or m is 4 to 38); an acyl group of a straight-chain or branched alkane am-dicarboxylic acid; CH₃(CH₂)ₚ((CH₂)_{q},COOH)CHNH—CO(CH₂)₂CO—, where p and q are integers and p+q is 8 to 33; CH₃(CH₂)ᵣCO—NHCH(COOH)(CH₂)₂CO—, where r is 10 to 24; CH₃(CH₂)ₛCO—NHCH((CH₂)₂COOH)CO—, where s is 8 to 24; COOH(CH₂)ₜCO—, where t is 8 to 24; —NHCH(COOH)(CH₂)₄NH—CO(CH₂)ᵤCH₃, where u is 8 to 18; —NHCH(COOH)(CH₂)₄NH—COCH((CH₂)₂COOH)NH—CO(CH₂)_{w}CH₃, where w is 10 to 16; —NHCH(COOH)(CH₂)₄NH—CO(CH₂)₂CH(COOH)NH—CO(CH₂)ₓCH₃, where x is 10 to 16; or —NHCH(COOH)(CH₂)₄NH—CO(CH₂)₂CH(COOH)NHCO(CH₂)_{y}CH₃, where y is 1 to 22.

In other embodiments, the polypeptide therapeutic is modified by addition of a chemically reactive group such as a maleimide group, as described in U.S. Pat. No. 6,593,295. These groups can react with available reactive functionalities on blood components to form covalent bonds and can extending the effective therapeutic in vivo half-life of the modified insulinotropic peptides. To form covalent bonds with the functional group on a protein, one can use as a chemically reactive group a wide variety of active carboxyl groups (e.g., esters) where the hydroxyl moiety is physiologically acceptable at the levels required to modify the polypeptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA).

Primary amines are the principal targets for NHS esters. Accessible α-amine groups present on the N-termini of proteins and the ε-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the invention, the functional group on the protein will be a thiol group and the chemically reactive group will be a maleimido-containing group such as gamma-maleimide-butrylamide (GMBA or MPA). Such maleimide containing groups are referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl is formed, which cannot be cleaved under physiological conditions.

### Peptide Vectors

The compounds of the invention can feature any of polypeptides described herein, for example, any of the peptides described in Table 1 (e.g., Angiopep-1 or Angiopep-2), or a fragment or analog thereof. In certain embodiments, the polypeptide may have at least 35%, 40%, 50%, 60%, 70%, 80%, 90%, 95%, 99%, or even 100% identity to a polypeptide described herein. The polypeptide may have one or more (e.g., 2, 3, 4, 5, 6, 7, 8, 9, 10, 11, 12, 13, 14, or 15) substitutions relative to one of the sequences described herein. Other modifications are described in greater detail below.

The invention also features fragments of these polypeptides (e.g., a functional fragment). In certain embodiments, the fragments are capable of efficiently being transported to or accumulating in a particular cell type (e.g., liver, eye, lung, kidney, or spleen) or are efficiently transported across the BBB. Truncations of the polypeptide may be 1, 2, 3, 4, 5, 6, 7, 8, 9, 10, 11, 12, or more amino acids from either the N-terminus of the polypeptide, the C-terminus of the polypeptide, or a combination thereof. Other fragments include sequences where internal portions of the polypeptide are deleted.

Additional polypeptides may be identified by using one of the assays or methods described herein. For example, a candidate polypeptide may be produced by conventional peptide synthesis, conjugated with paclitaxel and administered to a laboratory animal. A biologically-active polypeptide conjugate may be identified, for example, based on its ability to increase survival of an animal injected with tumor cells and treated with the conjugate as compared to a control which has not been treated with a conjugate (e.g., treated with the unconjugated agent). For example, a biologically active polypeptide may be identified based on its location in the parenchyma in an in situ cerebral perfusion assay.

Assays to determine accumulation in other tissues may be performed as well. Labelled conjugates of a polypeptide can be administered to an animal, and accumulation in different organs can be measured. For example, a polypeptide conjugated to a detectable label (e.g., a near-IR fluorescence spectroscopy label such as Cy5.5) allows live in vivo visualization. Such a polypeptide can be administered to an animal, and the presence of the polypeptide in an organ can be detected, thus allowing determination of the rate and amount of accumulation of the polypeptide in the desired organ. In other embodiments, the polypeptide can be labelled with a radioactive isotope (e.g., ¹²⁵I). The polypeptide is then administered to an animal. After a period of time, the animal is sacrificed and the organs are extracted. The amount of radioisotope in each organ can then be measured using any means known in the art. By comparing the amount of a labeled candidate polypeptide in a particular organ relative to the amount of a labeled control polypeptide, the ability of the candidate polypeptide to access and accumulate in a particular tissue can be ascertained. Appropriate negative controls include any peptide or polypeptide known not to be efficiently transported into a particular cell type (e.g., a peptide related to Angiopep that does not cross the BBB, or any other peptide).

Additional sequences are described in U.S. Pat. No. 5,807,980 (e.g., SEQ ID NO:102 herein), 5,780,265 (e.g., SEQ ID NO:103), 5,118,668 (e.g., SEQ ID NO:105). An exemplary nucleotide sequence encoding an aprotinin analog atgagaccag atttctgcct cgagccgccg tacactgggc cctgcaaagc tcgtatcatc cgttacttct acaatgcaaa ggcaggcctg tgtcagacct tcgtatacgg cggctgcaga gctaagcgta acaacttcaa atccgcggaa gactgcatgc gtacttgcgg tggtgcttag; SEQ ID NO:6; Genbank accession No. X04666). Other examples of aprotinin analogs may be found by performing a protein BLAST (Genbank: www.ncbi.nlm.nih.gov/BLAST/) using the synthetic aprotinin sequence (or portion thereof) disclosed in International Application No. PCT/CA2004/000011. Exemplary aprotinin analogs are also found under accession Nos. CAA37967 (GI:58005) and 1405218C (G1:3604747).

### Modified Polypeptides

The peptide vectors and polypeptide therapeutics used in the invention may have a modified amino acid sequence. In certain embodiments, the modification does not destroy significantly a desired biological activity (e.g., ability to cross the BBB or GLP-1 agonist activity). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified peptide vector or polypeptide therapeutic may have or may optimize a characteristic of a polypeptide, such as in vivo stability, bioavailability, toxicity, immunological activity, immunological identity, and conjugation properties.

Modifications include those by natural processes, such as posttranslational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from posttranslational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy terminus of any of the polypeptides of the invention can facilitate conjugation of these polypeptides by, e.g., disulfide bonding. For example, Angiopep-1 (SEQ ID NO:67), Angiopep-2 (SEQ ID NO:97), or Angiopep-7 (SEQ ID NO:112) can be modified to include a single cysteine residue at the amino-terminus (SEQ ID NOS: 71, 113, and 115, respectively) or a single cysteine residue at the carboxy-terminus (SEQ ID NOS: 72, 114, and 116, respectively). Amino acid substitutions can be conservative (i.e., wherein a residue is replaced by another of the same general type or group) or non-conservative (i.e., wherein a residue is replaced by an amino acid of another type). In addition, a non-naturally occurring amino acid can be substituted for a naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 2. If such substitutions result in a change not desired, then other type of substitutions, denominated “exemplary substitutions” in Table 3, or as further described herein in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),
(2) neutral hydrophilic: Cysteine (Cys), Serine (Ser), Threonine (Thr)
(3) acidic/negatively charged: Aspartic acid (Asp), Glutamic acid (Glu)
(4) basic: Asparagine (Asn), Glutamine (Gin), Histidine (His), Lysine (Lys), Arginine (Arg)
(5) residues that influence chain orientation: Glycine (Gly), Proline (Pro);
(6) aromatic: Tryptophan (Trp), Tyrosine (Tyr), Phenylalanine (Phe), Histidine (His),
(7) polar: Ser, Thr, Asn, Gln
(8) basic positively charged: Arg, Lys, His, and;
(9) charged: Asp, Glu, Arg, Lys, His
   Other amino acid substitutions are listed in Table 3.

TABLE 2
Amino acid substitutions

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Polypeptide Derivatives and Peptidomimetics

In addition to polypeptides consisting of naturally occurring amino acids, peptidomimetics or polypeptide analogs are also encompassed by the present invention and can form the peptide vectors or polypeptide therapeutics used in the compounds of the invention. Polypeptide analogs are commonly used in the pharmaceutical industry as non-peptide drugs with properties analogous to those of the template polypeptide. The non-peptide compounds are termed “peptide mimetics” or peptidomimetics (Fauchere et al., Infect. Immun. 54:283-287, 1986 and Evans et al., J. Med. Chem. 30:1229-1239, 1987). Peptide mimetics that are structurally related to therapeutically useful peptides or polypeptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH—(cis and trans), —CH₂SO—, —CH(OH)CH₂—, —COCH₂—etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and others.

While the peptide vectors described herein may efficiently cross the BBB or target particular cell types (e.g., those described herein), their effectiveness may be reduced by the presence of proteases. Likewise, the effectiveness of polypeptide therapeutics used in the invention may be similarly reduced. Serum proteases have specific substrate requirements, including L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). In light of this, it is often advantageous to use modified versions of polypeptides. The modified polypeptides retain the structural characteristics of the original L-amino acid polypeptides, but advantageously are not readily susceptible to cleavage by protease and/or exopeptidases.

Systematic substitution of one or more amino acids of a consensus sequence with D-amino acid of the same type (e.g., an enantiomer; D-lysine in place of L-lysine) may be used to generate more stable polypeptides. Thus, a polypeptide derivative or peptidomimetic as described herein may be all L-, all D-, or mixed D, L polypeptides. The presence of an N-terminal or C-terminal D-amino acid increases the in vivo stability of a polypeptide because peptidases cannot utilize a D-amino acid as a substrate (Powell et al., Pharm. Res. 10:1268-1273, 1993). Reverse-D polypeptides are polypeptides containing D-amino acids, arranged in a reverse sequence relative to a polypeptide containing L-amino acids. Thus, the C-terminal residue of an L-amino acid polypeptide becomes N-terminal for the D-amino acid polypeptide, and so forth. Reverse D-polypeptides retain the same tertiary conformation and therefore the same activity, as the L-amino acid polypeptides, but are more stable to enzymatic degradation in vitro and in vivo, and thus have greater therapeutic efficacy than the original polypeptide (Brady and Dodson, Nature 368:692-693, 1994 and Jameson et al., Nature 368:744-746, 1994). In addition to reverse-D-polypeptides, constrained polypeptides comprising a consensus sequence or a substantially identical consensus sequence variation may be generated by methods well known in the art (Rizo et al., Ann. Rev. Biochem. 61:387-418, 1992). For example, constrained polypeptides may be generated by adding cysteine residues capable of forming disulfide bridges and, thereby, resulting in a cyclic polypeptide. Cyclic polypeptides have no free N- or C-termini. Accordingly, they are not susceptible to proteolysis by exopeptidases, although they are, of course, susceptible to endopeptidases, which do not cleave at polypeptide termini. The amino acid sequences of the polypeptides with N-terminal or C-terminal D-amino acids and of the cyclic polypeptides are usually identical to the sequences of the polypeptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197, 1996). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the polypeptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular —S-alkyl bonds can be prepared by conventional solid phase chemistry while incorporating an amino acid residue with a suitable amino-protected side chain, and a suitable S-protected cysteine or homocysteine residue at the position selected for cyclization.

Another effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharm. Res. 10:1268-1273, 1993). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified polypeptides consisting of polypeptides bearing an N-terminal acetyl group and/or a C-terminal amide group.

Also included by the present invention are other types of polypeptide derivatives containing additional chemical moieties not normally part of the polypeptide, provided that the derivative retains the desired functional activity of the polypeptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—OC—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives.

Longer polypeptide sequences which result from the addition of additional amino acid residues to the polypeptides described herein are also encompassed in the present invention. Such longer polypeptide sequences can be expected to have the same biological activity and specificity (e.g., cell tropism) as the polypeptides described above. While polypeptides having a substantial number of additional amino acids are not excluded, it is recognized that some large polypeptides may assume a configuration that masks the effective sequence, thereby preventing binding to a target (e.g., a member of the OB receptor family). These derivatives could act as competitive antagonists. Thus, while the present invention encompasses polypeptides or derivatives of the polypeptides described herein having an extension, desirably the extension does not destroy the cell targeting activity of the polypeptides or its derivatives.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids shared with one of the described polypeptides which desirably results in a chimeric or fusion protein that has an equivalent or greater functional activity.

Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action, and better bioavailability.

Peptidomimetics compounds can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12:145, 1997). Examples of methods for the synthesis of molecular libraries can be found in the art, for example, in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carell et al. (Angew. Chem., Int. Ed. Engl. 33:2059, 1994 and ibid 2061); and in Gallop et al. (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it can be isolated and purified by any number of standard methods including, but not limited to, differential solubility (e.g., precipitation), centrifugation, chromatography (e.g., affinity, ion exchange, and size exclusion), or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000 and Hanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties. Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays amenable to automation.

### Linkers

The polypeptide therapeutic (e.g., leptin) may be bound to the vector peptide either directly (e.g., through a covalent bond such as a peptide bond) or may be bound through a linker. Linkers include chemical linking agents (e.g., cleavable linkers) and peptides.

In some embodiments, the linker is a chemical linking agent. The polypeptide therapeutic and vector peptide may be conjugated through sulfhydryl groups, amino groups (amines), and/or carbohydrates or any appropriate reactive group. Homobifunctional and heterobifunctional cross-linkers (conjugation agents) are available from many commercial sources. Regions available for cross-linking may be found on the polypeptides of the present invention. The cross-linker may comprise a flexible arm, e.g., 2, 3, 4, 5, 6, 7, 8, 9, 10, 11, 12, 13, 14, or 15 carbon atoms. Exemplary cross-linkers include BS3 ([Bis(sulfosuccinimidyl)suberate]; BS3 is a homobifunctional N-hydroxysuccinimide ester that targets accessible primary amines), NHS/EDC(N-hydroxysuccinimide and N-ethyl-(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-5-acetylthioacetate; SATA is reactive towards amines and adds protected sulfhydryls groups).

To form covalent bonds, one can use as a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA).

Primary amines are the principal targets for NHS esters. Accessible α-amine groups present on the N-termini of proteins and the ε-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the invention, the functional group on the protein will be a thiol group and the chemically reactive group will be a maleimido-containing group such as gamma-maleimide-butrylamide (GMBA or MPA). Such maleimide containing groups are referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

In other embodiments, the linker includes at least one amino acid (e.g., a peptide of at least 2, 3, 4, 5, 6, 7, 10, 15, 20, 25, 40, or 50 amino acids). In certain embodiments, the linker is a single amino acid (e.g., any naturally occurring amino acid such as Cys). In other embodiments, a glycine-rich peptide such as a peptide having the sequence [Gly-Gly-Gly-Gly-Ser]ₙ where n is 1, 2, 3, 4, 5 or 6 is used, as described in U.S. Pat. No. 7,271,149. In other embodiments, a serine-rich peptide linker is used, as described in U.S. Pat. No. 5,525,491. Serine rich peptide linkers include those of the formula [X-X-X-X-Gly]_{y}, where up to two of the X are Thr, and the remaining X are Ser, and y is 1 to 5 (e.g., Ser-Ser-Ser-Ser-Gly, where y is greater than 1). In some cases, the linker is a single amino acid (e.g., any amino acid, such as Gly or Cys).

Examples of suitable linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. In one particular embodiment, the further linker is succinic acid which, e.g., forms an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a N^{ε}-acylated lysine residue.

### Metabolic Disorder Therapy

In certain embodiments, the conjugate of the invention is used to treat a metabolic disorder. Such disorders include diabetes (type I or type II), obesity, hyperglycemia, dyslipidemia, hypertriglyceridemia, syndrome X, insulin resistance, IGT, diabetic dyslipidemia, hyperlipidemia, a cardiovascular disease, and hypertension. Leptin decreases food intake and thus can be used to reduce weight and to treat diseases where reduced food intake or weight loss is beneficial.

### Neurological Disease Therapy

Because polypeptides described herein are capable of transporting an agent across the BBB, the compounds of the invention are also useful for the treatment of neurological diseases such as neurodegenerative diseases or other conditions of the central nervous system (CNS), the peripheral nervous system, or the autonomous nervous system (e.g., where neurons are lost or deteriorate). Many neurodegenerative diseases are characterized by ataxia (i.e., uncoordinated muscle movements) and/or memory loss. Neurodegenerative diseases include Alexander disease, Alper disease, Alzheimer's disease, amyotrophic lateral sclerosis (ALS; i.e., Lou Gehrig's disease), ataxia telangiectasia, Batten disease (Spielmeyer-Vogt-Sjogren-Batten disease), bovine spongiform encephalopathy (BSE), Canavan disease, Cockayne syndrome, corticobasal degeneration, Creutzfeldt-Jakob disease, Huntington's disease, HIV-associated dementia, Kennedy's disease, Krabbé disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Schilder's disease (i.e., adrenoleukodystrophy), schizophrenia, spinocerebellar ataxia, spinal muscular atrophy, Steele-Richardson, Olszewski disease, and tabes dorsalis.

### Additional Indications

The conjugates of the invention can also be used to treat diseases found in other organs or tissues. For example, Angiopep-7 (SEQ ID NO:112) is efficiently transported into liver, lung, kidney, spleen, and muscle cells, allowing for the preferential treatment of diseases associated with these tissues (e.g., hepatocellular carcinoma and lung cancer). The compounds of the presents invention may also be used to treat genetic disorders, such as Down syndrome (i.e., trisomy 21), where down-regulation of particular gene transcripts may be useful.

### Administration and Dosage

The present invention also features pharmaceutical compositions that contain a therapeutically effective amount of a compound of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that comprise the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a subject with a clinically determined predisposition or increased susceptibility to a metabolic disorder or neurological disease. Compositions of the invention can be administered to the subject (e.g., a human) in an amount sufficient to delay, reduce, or preferably prevent the onset of clinical disease. In therapeutic applications, compositions are administered to a subject (e.g., a human) already suffering from disease (e.g., a metabolic disorder such as those described herein, or a neurological disease) in an amount sufficient to cure or at least partially arrest the symptoms of the condition and its complications. An amount adequate to accomplish this purpose is defined as a “therapeutically effective amount,” an amount of a compound sufficient to substantially improve some symptom associated with a disease or a medical condition. For example, in the treatment of a metabolic disorder (e.g., those described herein), an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Leptin may be administered at a dosage of anywhere from 0.001-3 mg/kg (e.g., .0.005, 0.01, 0.05, 0.1, 0.5, 1, 2, or 3 mg/kg). The compounds of the present invention may be administered in equivalent doses of as specified for leptin, may be administered in higher equivalent doses (e.g., 10%, 25%, 50%, 100%, 200%, 500%, 1000% greater doses), or can be administered in lower equivalent doses (e.g., 90%, 75%, 50%, 40%, 30%, 20%, 15%, 12%, 10%, 8%, 7%, 6%, 5%, 4%, 3%, 2%, 1%, 0.5%, or 0.1% of the equivalent dose). Amounts effective for this use may depend on the severity of the disease or condition and the weight and general state of the subject. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6,8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of the invention and used in the methods of this invention applied to mammals (e.g., humans) can be determined by the ordinarily-skilled artisan with consideration of individual differences in age, weight, and the condition of the subject. Because certain compounds of the invention exhibit an enhanced ability to cross the BBB, the dosage of the compounds of the invention can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated leptin, leptin analog, or OB receptor agonist. The agents of the invention are administered to a subject (e.g. a mammal, such as a human) in an effective amount, which is an amount that produces a desirable result in a treated subject (e.g. reduction in glycemia, reduced weight gain, increased weight loss, and reduced food intake). Therapeutically effective amounts can also be determined empirically by those of skill in the art.

The subject may also receive an agent in the range of about 80 μg to 240 mg equivalent dose as compared to leptin per dose one or more times per week (e.g., 2, 3, 4, 5, 6, or 7 or more times per week), 1 mg to 24 mg equivalent dose per day.

Single or multiple administrations of the compositions of the invention comprising an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

The compounds of the present invention may be used in combination with either conventional methods of treatment or therapy or may be used separately from conventional methods of treatment or therapy.

When the compounds of this invention are administered in combination therapies with other agents, they may be administered sequentially or concurrently to an individual. Alternatively, pharmaceutical compositions according to the present invention may be comprised of a combination of a compound of the present invention in association with a pharmaceutically acceptable excipient, as described herein, and another therapeutic or prophylactic agent known in the art.

### Example 1

### Synthesis of a Leptin Conjugate

The following procedure was used to generate a Leptin-(C11)-AN2 conjugate.

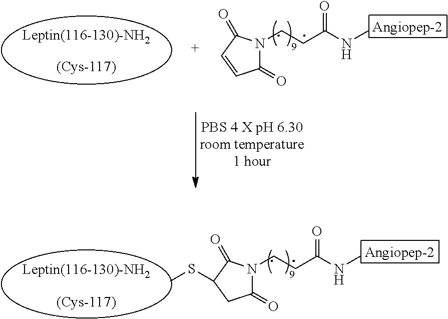

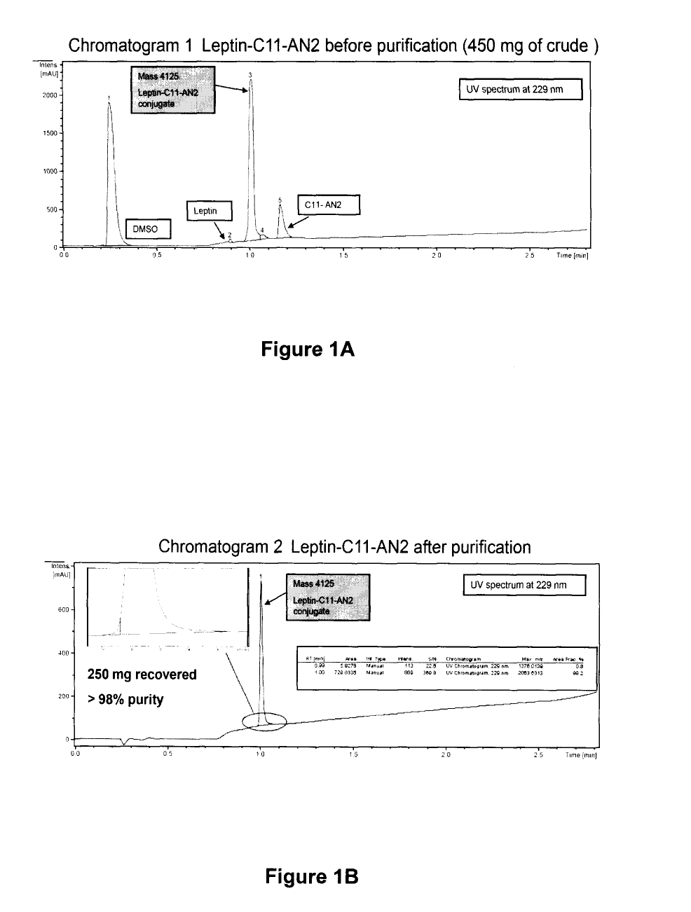

MUA-AN2 (264.6 mg, 91.5 mot, 1.2 eq., 82% peptide content) was dissolved in H₂O/ACN (9/1) (14 ml) by adjusting pH from 3.9 to 5.00 with addition of a 0.1 N NaOH solution (1.5 ml). This solution was added to a solution of Leptin₁₁₆₋₁₃₀-NH₂ (156.5 mg, 76.2 μmol, 1 eq., 76% peptide content) in PBS 4×(pH 6.61, 7 mL). Monitoring of the reaction was done with the analytical method described below. Results are shown in FIGS. 1A and 1B (chromatograms 1 and 2).

A cloudy suspension was observed as the reaction went to completion. After 1 h at room temperature, the reaction (3.62 mM) was complete and the mixture was purified immediately by FPLC chromatography (AKTAexplorer, see chromatogram 3, Table 1). Purification was performed on a GE Healthcare AKTA explorer column (GE Healthcare) 30 RPC resin (polystyrene/divinylbenzene), 95 ml, sample load: 450 mg in reaction buffer (21 ml), 10% ACN in H₂O, 0.05% TFA (60 ml), DMSO.HCl (pH 2.87, 6 ml), Solution A: H₂O, 0.05% TFA, Solution B: ACN, 0.05% TFA, Flow: 5-17 ml/min, Gradient: 10-30% B.

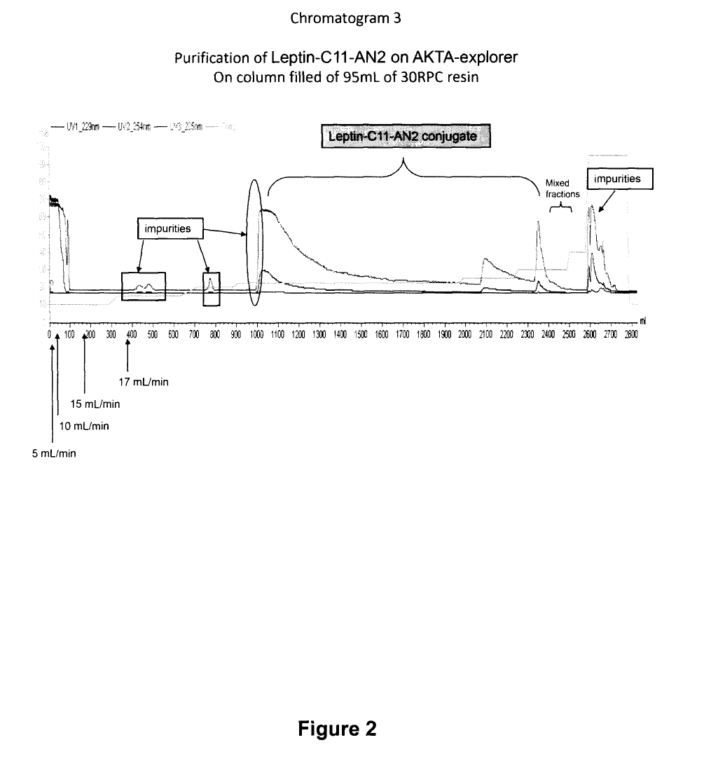

Purification results are shown in FIG. 2 (chromatogram 3). The gradient used to purify the compound is shown in the table below.

| Volume | Column | Flow rate | | |
|---|---|---|---|---|
| (ml) | volume (C.V.) | (ml/min) | % Solvent B | |
| 0 | 0 | 5 | 10 | |
| 33.58 | 0.35 | 10 | 10 | |
| 186.98 | 1.61 | 15 | 10 | |
| 282.51 | 1.01 | 15 | 15.0 | (over 3 min) |
| 346.26 | 0.67 | 16 | 15 | |
| 366.68 | 0.21 | 17 | 15 | |
| 625.3 | 2.72 | 17 | 20.0 | (over 5 min) |
| 876.28 | 2.64 | 17 | 22.5 | (over 2 min) |
| 1970.49 | 11.52 | 17 | 25.0 | (over 1 min) |
| 2233.45 | 2.77 | 17 | 30.0 | (over 1 min) |
| 2488.68 | 2.69 | 17 | 40.0 | (over 0.5 min) |
| 2577.28 | 0.93 | 17 | 95.0 | (over 1 min) |
| 2777.41 | 2.11 | 17 | 10.0 | (over 0.5 min) |

After evaporation of acetonitrile and lyophilization, a white solid (250 mg, 79%, purity>98%) was obtained. The mass was checked by ESI-TOFMS (Bruker Daltonics). To avoid the possibility that some remaining Leptin(116-130)-NH₂ might dimerize (≦5%, cysteine peptide Mw=3119.44), immediate purification was performed and an 1.2 equivalent excess of maleimido-(C11)-AN2 was used.

To monitor the reaction, the following analytical method was used. A Waters Acquity HPLC system with a Waters Acquity HPLC BEH phenyl column was used (1.7 μm, 2.1×50 mm). Detection was performed at 229 nm. Solution A was H₂O, 0.1% FA, and Solution B was acetonitrile (ACN), 0.1% formic acid (FA). Flow and gradient are shown in the Table below.

| Time | Flow | | | |
|---|---|---|---|---|
| (min) | (ml/min) | % A | % B | Curve |
| | 0.5 | 90 | 10 | |
| 0.4 | 0.5 | 90 | 10 | 6 |
| 0.7 | 0.5 | 70 | 30 | 6 |
| 2.2 | 0.5 | 30 | 70 | 6 |
| 2.4 | 0.5 | 10 | 90 | 6 |
| 2.7 | 0.5 | 10 | 90 | 6 |
| 2.8 | 0.5 | 90 | 10 | 6 |
| 2.81 | 0.5 | 90 | 10 | 6 |

From mass spectroscopy (ESI-TOF-MS; Bruker Daltonics): calculated 4125.53; found 4125.06, m/z 1376.01 (+3), 1032.26 (+4), 826.02 (+5), 688.52 (+6).

The conjugate was stored under nitrogen atmosphere, in a dark room, below −20° C.

The leptin conjugate generated using the procedure is called Leptin-AN2 (C11), due its 11-carbon linker. Other length carbon linker conjugates, were also generated, including Leptin-AN2 (C3) and Leptin AN2 (C6) using similar procedures.

### Example 2

### In situ Brain Perfursion of Leptin₁₁₆₋₁₃₀ Angiopep-2 Conjugates

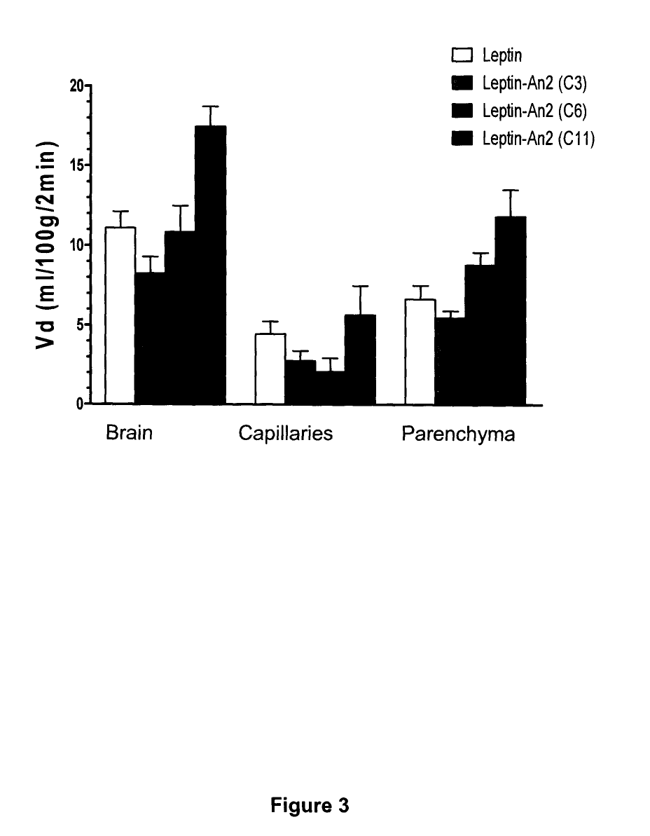

To determine which of the leptin conjugates most effectively crossed the blood-brain barrier, we tested each conjugate in the in situ brain perfusion assay. This assay is or a similar assay is described, for example, in U.S. Patent Publication No. 20060189515, which was based on a method described in Dagenais et al., 2000, J. Cereb. Blood Flow Metab. 20(2):381-386. The BBB transport constants were determined as previously described by Smith (1996, Pharm. Biotechnol. 8:285-307). From these experiments, Leptin-AN2 (C11) exhibed the greatest transport across the BBB as compared to the conjugates having C3 or a C6 linker and was thus selected for further experimentation (FIG. 3).

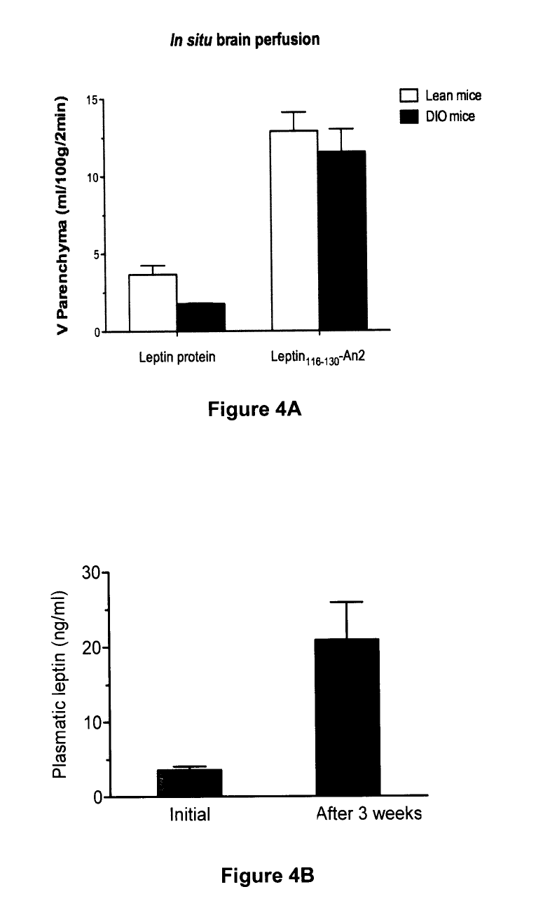

Transport of leptin was compared to the Leptin-AN2 (C11) conjugate using the in situ perfusion assay in lean and diet-induced obese (DIO) mice (available, e.g., from the Jackson laboratories). From these results, transport of leptin across the BBB in DIO mice was reduced as compared to in lean mice. By contrast, the Leptin-AN2 (C11) conjugate crossed the brain much more efficiently in both lean and DIO mice, and no statistically significant difference between the lean and DIO mice in transport of the conjugate was observed (FIG. 4A). Plasma leptin levels were observed to increase after 3 weeks on a high fat (60%) diet, suggesting that the mice were becoming leptin resistant (FIG. 4B).

### Example 3

### Effect of Leptin Conjugates on Food Intake and Weight Gain

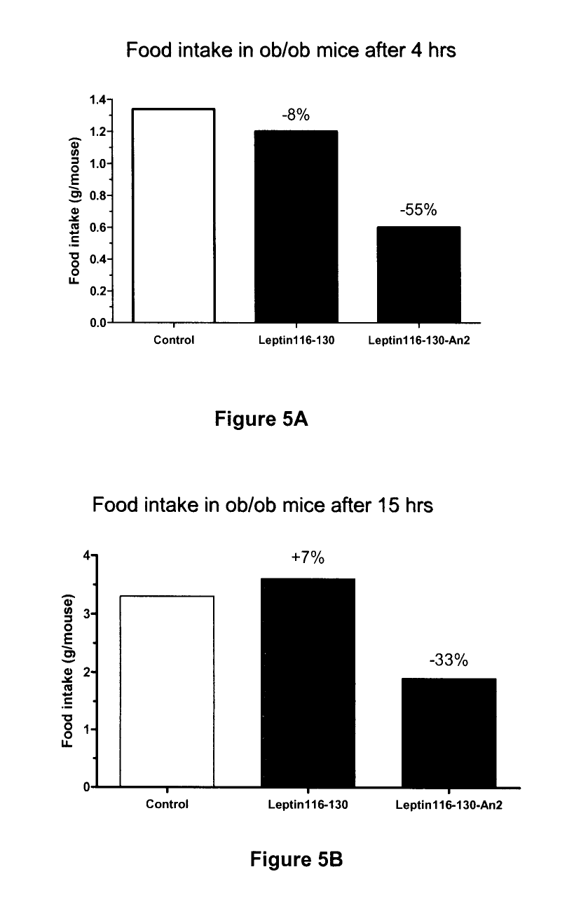

Mice were injected with an intravenous bolus of either Leptin-AN2 (C11) (eq. of 1 mg of leptin₁₁₆₋₁₃₀ per mouse), leptin₁₁₆₋₁₃₀ (1 mg/mouse), or a control (saline) (n=5 per group). Food intake of the mice was monitored at 4 hours (FIG. 5A) and at 15 hours (FIG. 5B). In both cases, the conjugate exhibited significantly greater reduction in food intake, as compared to either the control mice, or mice receiving leptin₁₁₆₋₁₃₀.

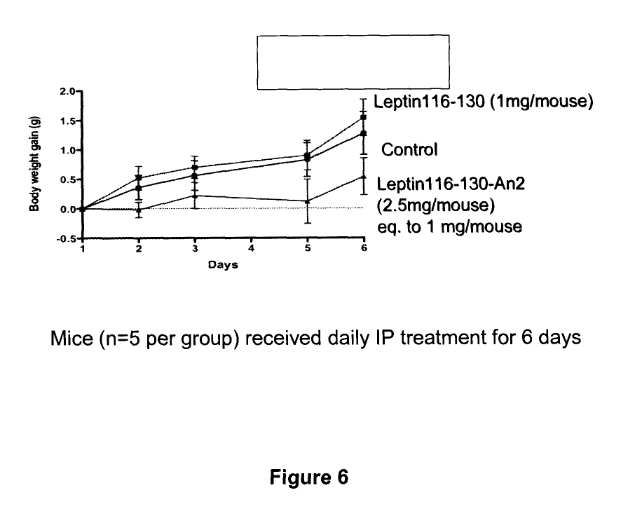

We also compared weight changes in DIO mice receiving the conjugate (2.5 mg/mouse; equivalent of 1 mg leptin₁₁₆₋₁₃₀ mg/mouse), leptin₁₁₆₋₁₃₀ (1 mg/mouse), and a control over a period of six days. Each mouse received daily treatment by intraperitoneal injection. Mice receiving leptin or the control exhibited similar amounts of weight gain over the six days, whereas mice receiving the conjugate showed marked reduction in weight gain (FIG. 6) as compared to the control mice and mice receiving leptin₁₁₆₋₁₃₀.

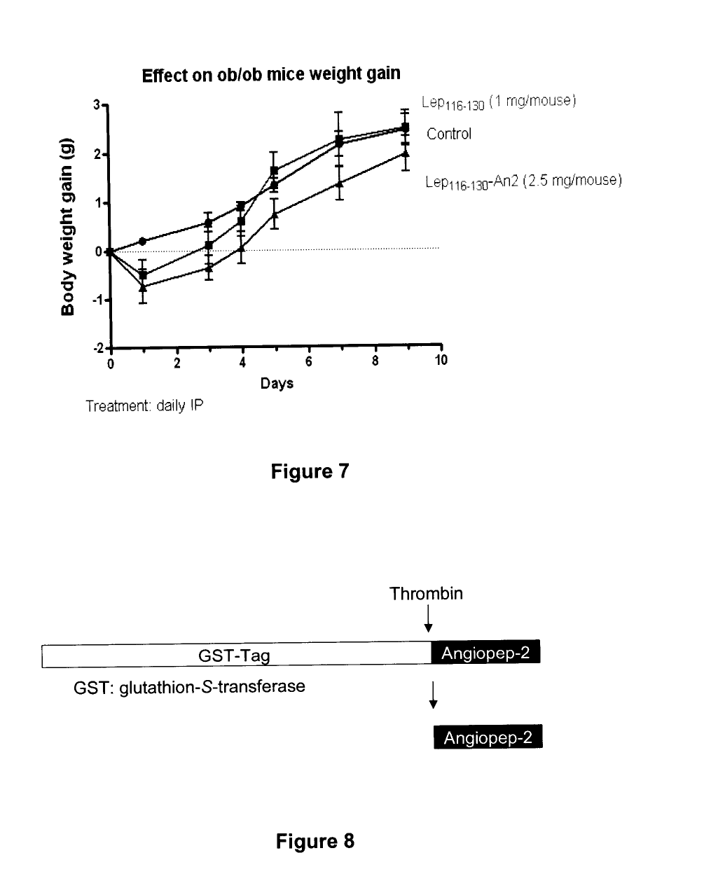

We further compared weight changes in leptin-deficient ob/ob mice receiving the conjugate (2.5 mg/mouse; equivalent of 1 mg leptin₁₁₆₋₁₃₀ mg/mouse), leptin₁₁₆₋₁₃₀ (1 mg/mouse), and a control over a period of six days. Each mouse (n=5 per group) received daily treatment by intraperitoneal injection. The mice receiving the conjugate exhibited lower weight gain than the mice receiving either leptin₁₁₆₋₁₃₀ or the control (FIG. 7) during the period of administration.

### Example 4

### Development of Recombinant Angiopep-2 and Angiopep-2 Leptin Fusion Proteins

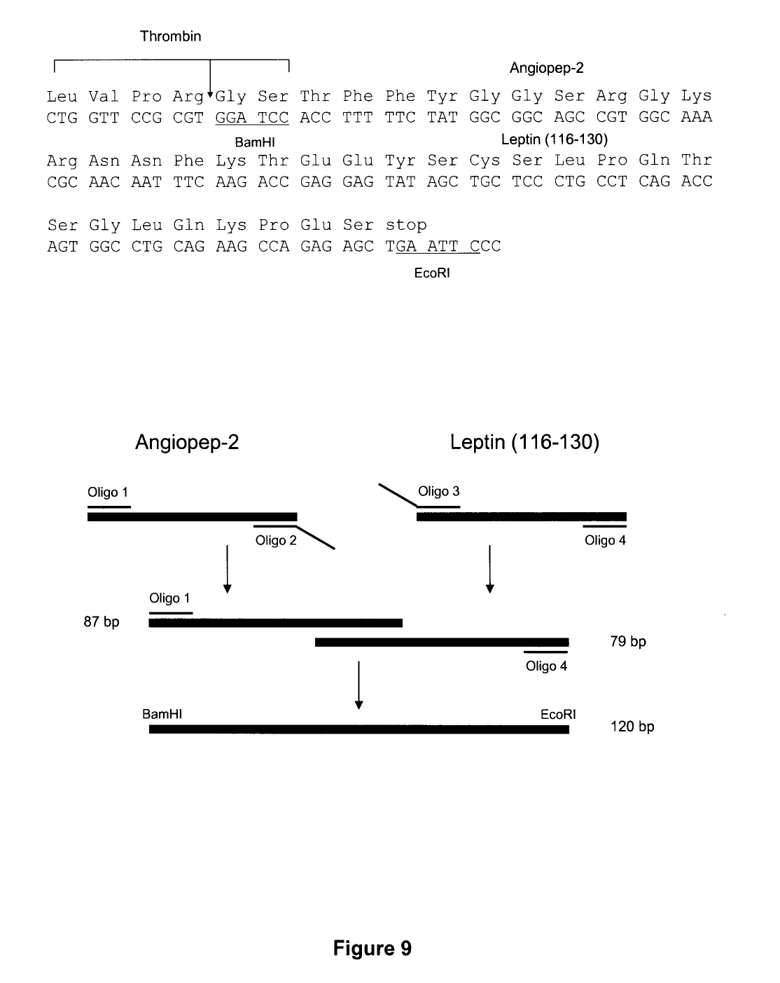

We also developed an Angiopep-2 fusion protein. As an initial step, a cDNA (ACC TTT TTC TAT GGC GGC AGC CGT GGC AAA CGC AAC AAT TTC AAG ACC GAG GAG TAT; SEQ ID NO:117) was created. This sequence was inserted into a pGEX vector system for bacterial expression, and sequence of the insert was verified (FIG. 8). The GST-Ant-Leptin₁₁₆₋₁₃₀ construct was made using an overlap extension PCR strategy (FIG. 9).

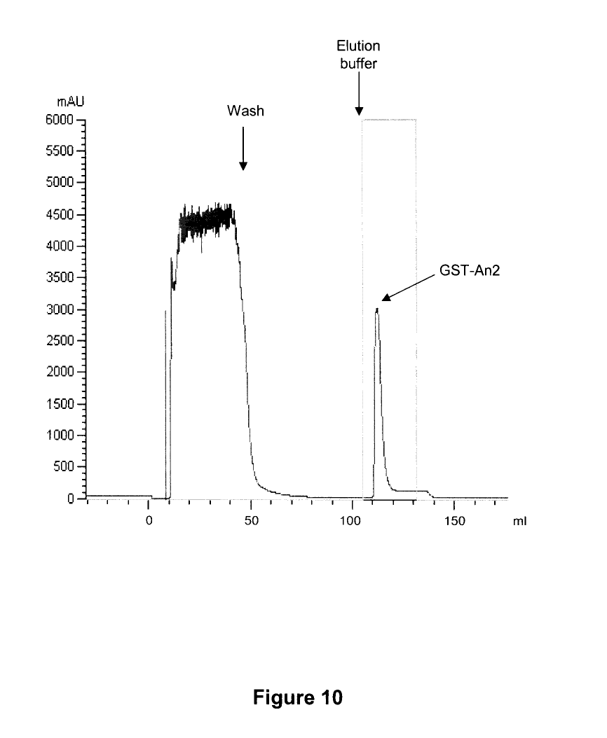

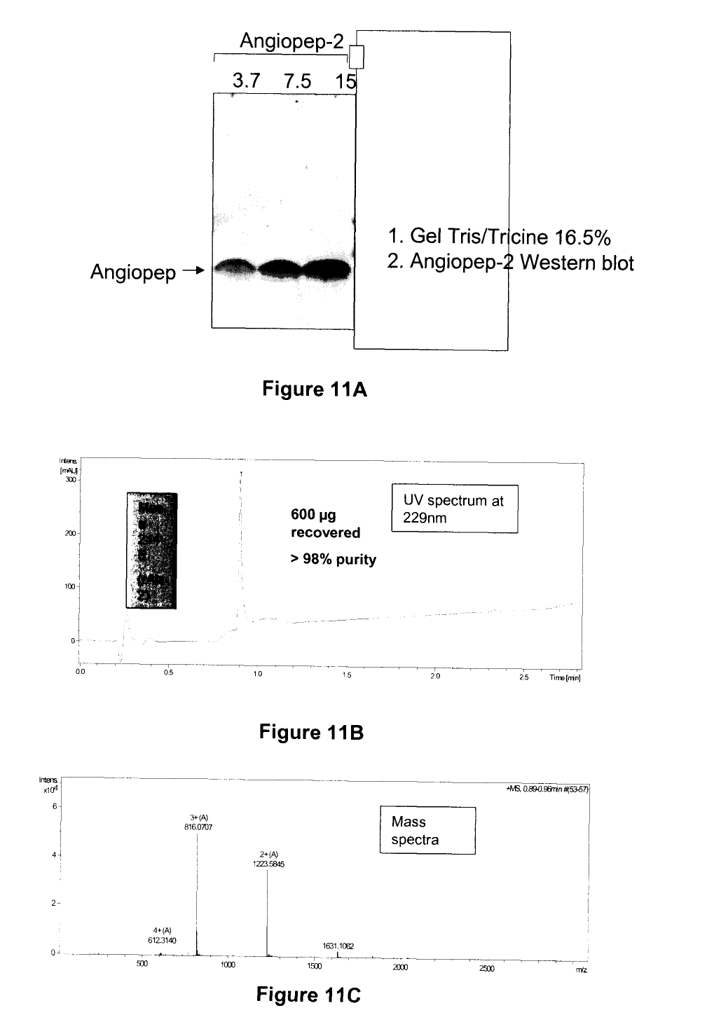

The recombinant Angiopep-2 was expressed in a bacterial expression system and purified using a GSH-Sepharose column. A chromatogram from this procedure is shown (FIG. 10). The purified Angiopep-2 was analyzed by Western blot using an Angiopep-2 antibody (FIG. 11A), by liquid chromatography (FIG. 11B), and by mass spectroscopy (FIG. 11C).

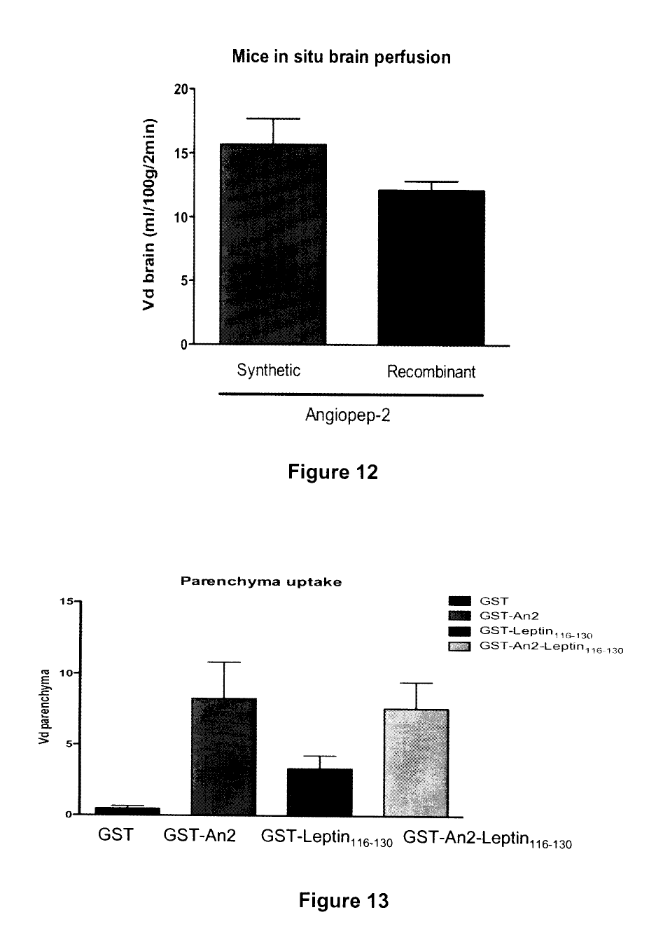

The in situ brain perfusion assay was performed using recombinant Angiopep-2. The results were compared to synthetic Angiopep-2 (FIG. 12). Similar levels of uptake were observed with both forms of Angiopep-2. Uptake into the parenchyma between GST, GST-Angiopep-2, GST-Leptin₁₁₆₋₁₃₀, and GST-Angiopep-2-Leptin₁₁₆₋₁₃₀ was compared (FIG. 13). These results show that fusion proteins containing the Angiopep-2 sequence are efficiently taken up into the parenchyma, whereas proteins lacking the Angiopep-2 sequence are taken up much less efficiently.

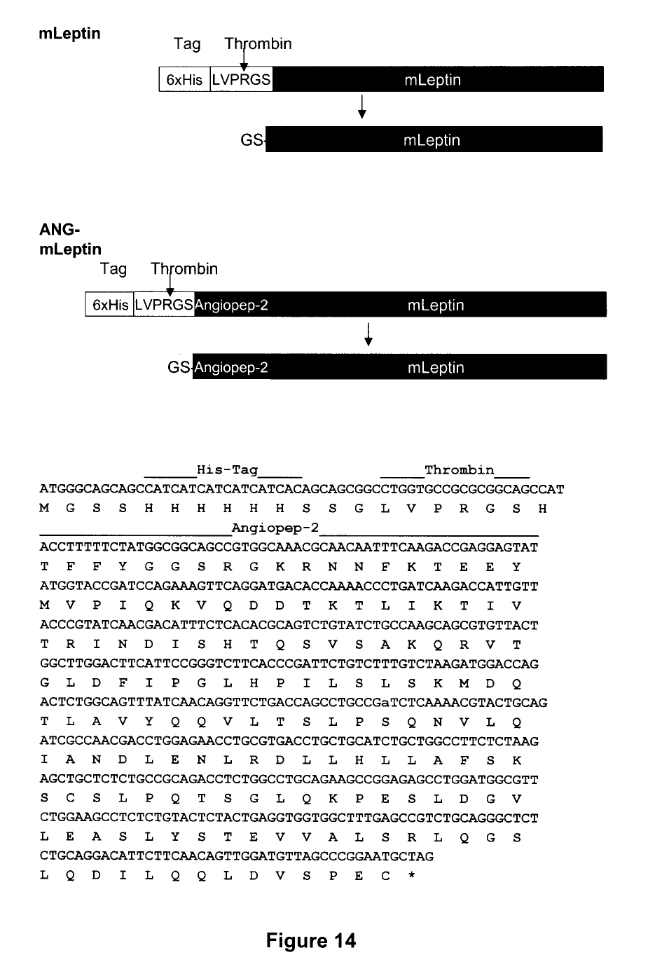

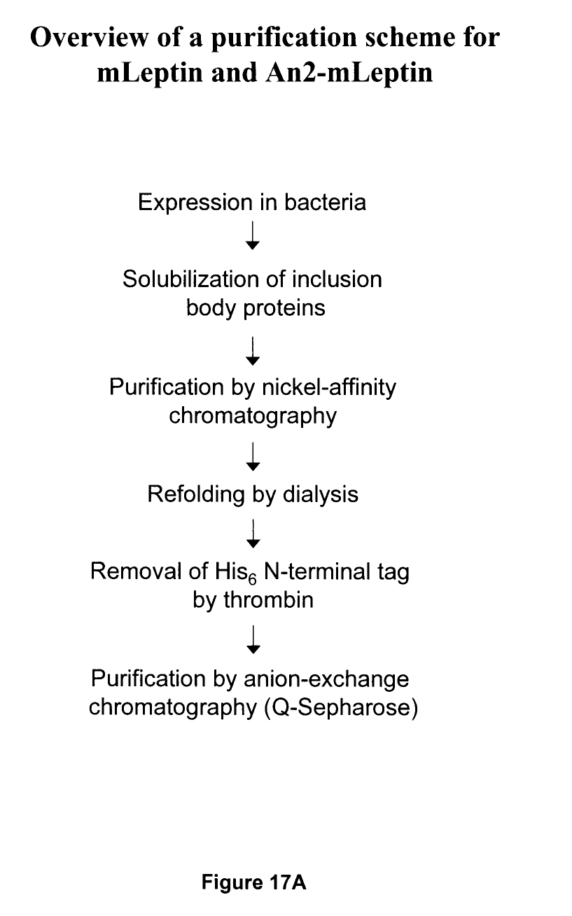

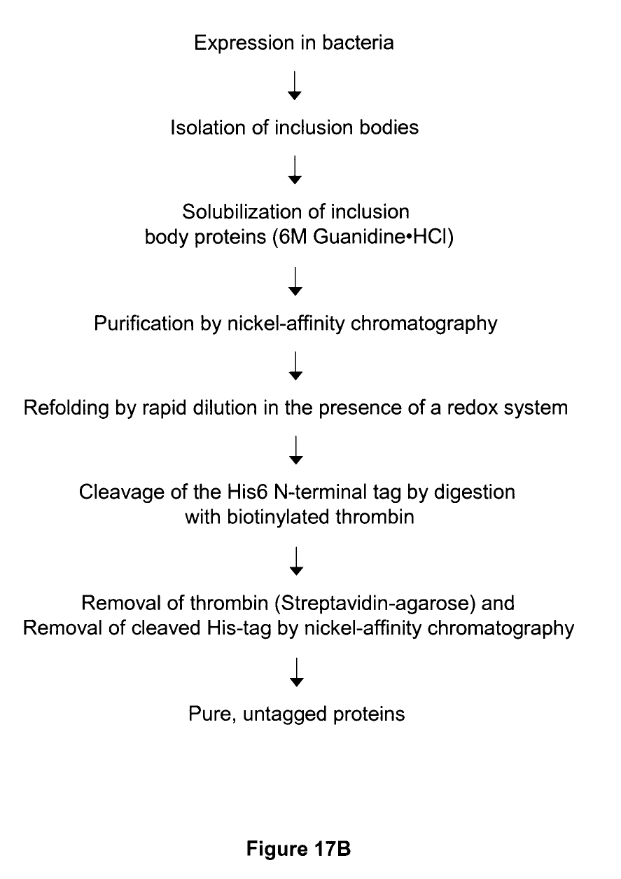

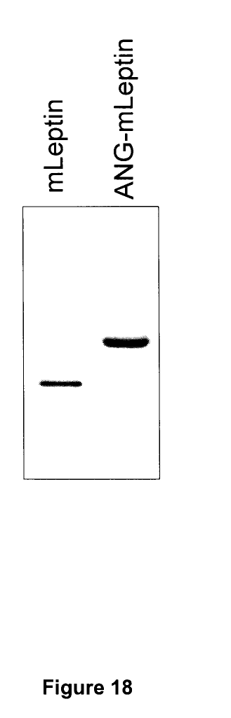

A His-tagged Angiopep-2/mouse leptin fusion protein containing the full length leptin sequence has been generated (FIG. 14). This fusion protein has been expressed in a bacterial expression system (FIG. 15). Exemplary purification schemes for the fusion protein are shown in FIGS. 17A and 17B. Results from a small scale purification are shown in FIG. 18.

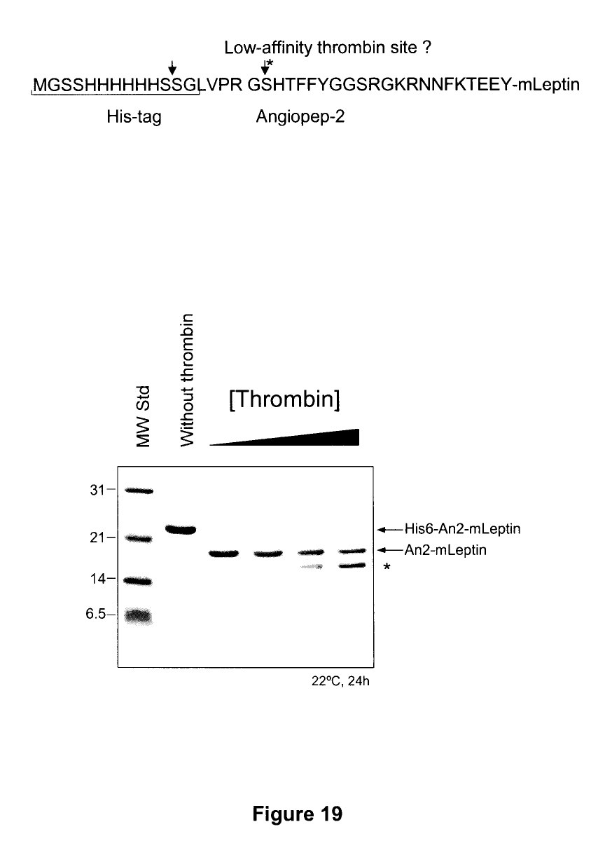

The thrombin cleavage step resulted in production of two products, suggesting the possibility that the Angiopep-2 sequence contains a low-affinity thrombin cleavage site, as shown in FIG. 19. As the leptin-Angiopep-2 has a propensity to agregate in solution, purification conditions to reduce the aggregation and improve yields are being tested.

### Example 5

### Brain Uptake and Activity of Leptin Fusion Proteins

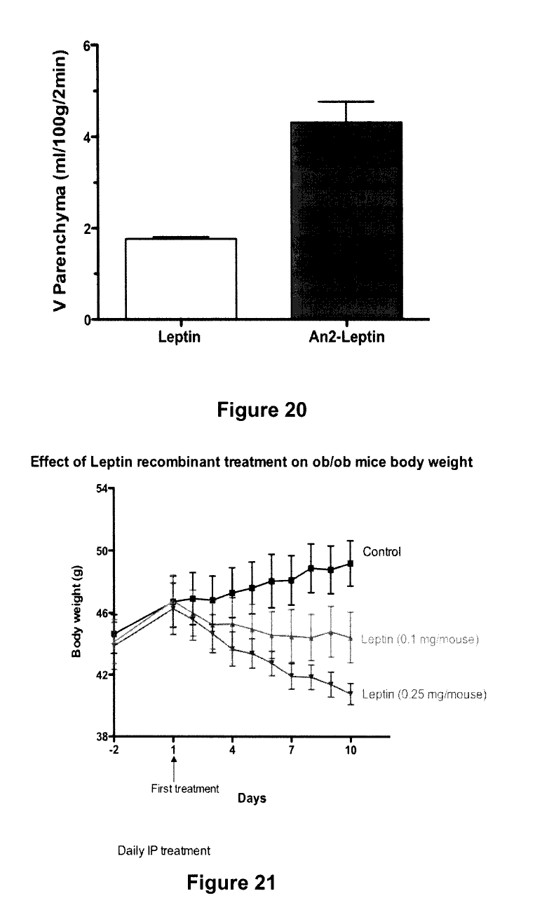

We then examined the ability of the Angiopep-2-leptin fusion protein to be taken up into the parenchyma of the brain of DIO mice as compared to leptin using the in situ brain perfusion assay (FIG. 20). From this experiment, we observed that the fusion protein exhibited increased uptake as compared to leptin.

As a control, we tested the ability of recombinant leptin to reduce body weight in ob/ob mice using either 0.1 mg/mouse or 0.25 mg/mouse daily. As shown in FIG. 21, leptin did indeed reduce body weight in these mice in a dose-dependent manner.

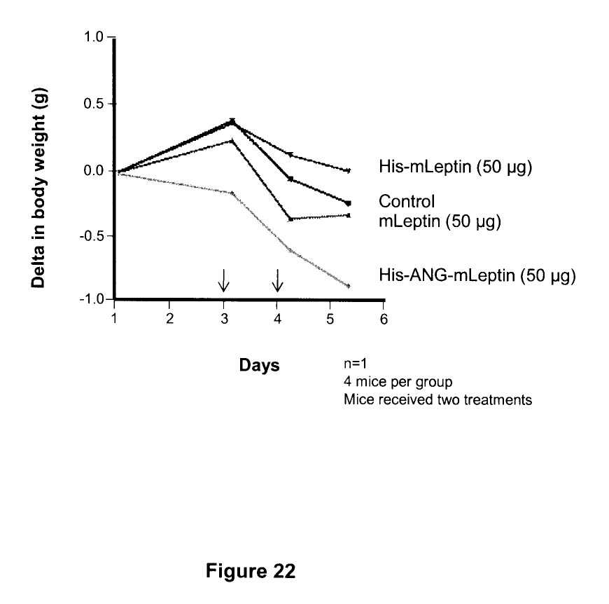

DIO mice were also treated with a control or with 50 μg his-tagged fusion protein, leptin, or the his-tagged leptin. Mice received two treatments, on days three and four as indicated. Based on these results, the greatest weight loss was observed in mice receiving the fusion protein (FIG. 22).

### Other Embodiments

All patents, patent applications, and publications mentioned in this specification are herein incorporated by reference, including U.S. Provisional Application Nos. 61/200,947 and 61/178,837, filed Dec. 5, 2008 and May 15, 2009, respectively, to the same extent as if each independent patent, patent application, or publication was specifically and individually indicated to be incorporated by reference.

What is claimed is:

1. A compound having the formula
A-X-B
wherein
A is a peptide vector comprising an amino acid sequence at least 70% identical to a sequence selected from the group consisting of SEQ ID NO:1-105 and 107-114, or a fragment thereof;
X is a linker; and
B is leptin, a leptin analog, or an OB receptor agonist.

2. The compound of claim 1, wherein A is a polypeptide having an amino acid sequence at least 70% identical to a sequence selected from the group consisting of Angiopep-1 (SEQ ID NO:67), Angiopep-2 (SEQ ID NO:97), cys-Angiopep-2 (SEQ ID NO:113), and Angiopep-2-cys (SEQ ID NO:114).

3. The compound of claim 2, wherein said sequence identity is at least 90%.

4. The compound of claim 3, wherein said polypeptide comprises an amino acid sequence selected from the group consisting of Angiopep-1 (SEQ ID NO:67), Angiopep-2 (SEQ ID NO:97), cys-Angiopep-2 (SEQ ID NO:113), and Angiopep-2-cys (SEQ ID NO:114).

5. The compound of claim 4, wherein said polypeptide consists of an amino acid sequence selected from the group consisting of Angiopep-1 (SEQ ID NO:67), Angiopep-2 (SEQ ID NO:97), cys-Angiopep-2 (SEQ ID NO:113), and Angiopep-2-cys (SEQ ID NO:114).

6. (canceled)

7. The compound of claim 1, wherein B comprises full-length human leptin, mature human leptin (amino acids 22-167 of the full length human leptin in FIG. 16), or leptin₁₁₆₋₁₃₀.

8. The compound of claim 1, wherein X has the formula: 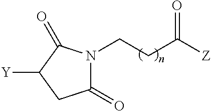
where n is an integer between 2 and 15; and either Y is a thiol on A and Z is a primary amine on B or Y is a thiol on B and Z is a primary amine on A.

9. The compound of claim 8, wherein n is 3, 6, or 11.

10. A compound having the structure:

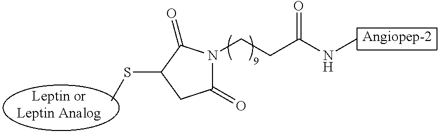

11. The compound of claim 10, wherein said leptin or leptin analog is full-length human leptin, mature human leptin (amino acids 22-167 of the full length human leptin), or leptin₁₁₆₋₁₃₀.

12. The compound of claim 1, wherein X is peptide bond.

13. The compound of claim 1, wherein X is at least one amino acid; and A and B are each covalently bonded to X by a peptide bond.

14. A nucleic acid molecule encoding the compound of claim 12.

15-17. (canceled)

18. A method of treating a subject having a metabolic disorder, said method comprising administering a compound of claim 1 in an amount sufficient to treat said disorder.

19. The method of claim 18, wherein said amount sufficient is less than 50% of the amount required for an equivalent dose of the leptin, leptin analog, or OB receptor agonist when not conjugated to the peptide vector.

20. The method of claim 19, wherein said amount is less than 15%.

21. The method of claim 18, wherein said metabolic disorder is diabetes, obesity, diabetes as a consequence of obesity, hyperglycemia, dyslipidemia, hypertriglyceridemia, syndrome X, insulin resistance, impaired glucose tolerance (IGT), diabetic dyslipidemia, hyperlipidemia, a cardiovascular disease, or hypertension.

22. The method of claim 18, wherein said disorder is diabetes, type II diabetes, or obesity.

23-24. (canceled)

25. A method of reducing food intake by, or reducing body weight of, a subject, said method comprising administering a compound of claim 1 to a subject in an amount sufficient to reduce food intake or reduce body weight.

26. The method of claim 25, wherein said subject is overweight or obese.

27. The method of claim 25, wherein said subject is bulimic.
